(19) United States
(12) Patent Application Publication
(10) Pub. No.: US 2015/0328474 A1
(43) Pub. Date: Nov. 19, 2015

(54) A SAFE SKIN TREATMENT APPARATUS FOR PERSONAL USE AND METHOD FOR ITS USE

(71) Applicant: SYNEROM MEDICAL LTD., Yoqneam Illit (IL)

(72) Inventors: Lion Flyash, Nazareth Illit (IL); Genady Nahson, Netanya (IL)

(73) Assignee: SYNERON MEDICAL LTD, Yoqneam Illit (IL)

(21) Appl. No.: 14/350,068

(22) PCT Filed: Nov. 19, 2012
(86) PCT No.: PCT/IL12/00375

§ 371 Date: Apr. 4, 2014

Related U.S. Application Data
(60) Provisional application No. 61/563,562, filed on Nov. 24, 2011.

(51) Int. Cl. A61N 1/40; A61N 7/00

(52) U.S. Cl. CPC A61N 1/403; A61N 7/00; A61N 2007/0034

(57) ABSTRACT

Disclosed is a method of controlling an applicator coupling skin heating energy to the skin. The skin heating energy is applied to the skin as a function of electrode-to-skin coupling quality. In cases where only partial electrode-to-skin contact is detected the skin heating energy is adjusted accordingly. Disclosed is also an apparatus for implementing this method.

### CROSS-REFERENCE TO RELATED APPLICATIONS

This is an application for a United States utility patent and is being filed as a national application in the United States Patent Office under 35 U.S.C. 371 and claims the benefit of the filing date of U.S. provisional application for patent that was filed on Nov. 24, 2011 and assigned Ser. No. 61/563,562 by being a national stage filing of International Application Number PCT/IL2012/000375 filed on Nov. 12, 2012, each of which are incorporated herein by reference in their entirety.

### TECHNOLOGY FIELD

The method and apparatus relate to the field of skin treatment and personal cosmetic procedures and, in particular, to safe skin treatment procedures.

### BACKGROUND

External appearance is important to practically everybody. In recent years, methods and apparatuses have been developed for different cosmetic treatments to improve external appearance. Among these are: hair removal, treatment of vascular lesions, wrinkle reduction, collagen destruction, circumference reduction, skin rejuvenation, and others. In these treatments, a volume of skin to be treated is heated to a temperature that is sufficiently high as to perform the treatment and produce one of the desired treatment effects. The treatment temperature is typically in the range of 38-60 degrees Celsius.

One method used for heating the epidermal and dermal layers of the skin is pulsed or continuous radio frequency (RF) energy. In this method, electrodes are applied to the skin and an RF voltage, in a continuous or pulse mode, is applied across the electrodes. The properties of the voltage are selected to generate an RF induced current in the skin to be treated. The current heats the skin to the required temperature and causes a desired effect, performing one or more of the listed above treatments.

Another method used for heating the epidermal and dermal layers of the skin is illuminating the skin segment to be treated by optical, typically infrared (IR) radiation. In this method, a segment of skin is illuminated by optical radiation in a continuous or pulse mode. The power of the radiation is set to produce a desired skin effect. The IR radiation heats the skin to the required temperature and causes one or more of the desired effects.

An additional method used for heating the epidermal and dermal layers of the skin is application of ultrasound energy to the skin. In this method, ultrasound transducers are coupled to the skin and ultrasound energy is applied to the skin between the transducers. The properties of the ultrasound energy are selected to heat a target volume of the skin (usually the volume between the electrodes) to a desired temperature, causing one or more of the desired treatment effects, which may be hair removal, collagen destruction, circumference reduction, skin rejuvenation, and others.

Methods exist which simultaneously apply a combination of one or more skin heating techniques to the skin. Since all of the methods alter the skin temperature, monitoring of the temperature is frequently used to control the treatment. In order to continuously monitor skin temperature, suitable sensors such as a thermocouple or a thermistor could be built into the electrodes or transducers through which the energy is applied to the skin. Despite the temperature monitoring, certain potential skin damage risk still exists, since the sensor response time depends on heat conductivity from the skin to the sensor and inside the sensor, and may be too long and even damaging to the skin before the sensor reduces or cuts off the skin heating power. To some extent, this risk can be avoided by reducing the cut-off temperature limit operating the sources of optical radiation, RF energy, and ultrasound energy. However, this would limit the RF energy transmitted to the skin and the treatment efficacy. In some instances, for example, when the applicator is static, the temperature of the skin (and of the electrodes) may increase fast enough to cause skin damage.

The devices delivering energy to the skin, such as electrodes, transducers and similar are usually packed in a convenient casing, an applicator, operative to be held and moved across the treated skin segment. The user has to adjust applicator movement speed to a given constant skin heating energy supply, such as to enable optimal or proper skin treatment. However, at present the user has no indication if the selected applicator speed is proper or not.

The skin is usually soft and good quality contact between RF electrodes and the skin can be achieved even in skin surface segments where the skin has curved topography. When solid and rigid electrodes are applied to a skin surface covering a “bony” area, having minimal fat and muscle tissue, such as for example, forehead, chin, and similar the contact between the RF electrode and skin becomes partial and the quality of the contact deteriorates and it becomes improper or insufficient for skin treatment. When the quality of the contact deteriorates the current density in the remaining contact points grows fast and could cause skin burns.

### BRIEF SUMMARY

When heating energy is applied to a segment of skin to be treated and the applicator is displaced from one segment of skin to another, there is a difference in the rate of the skin temperature increase or change, which depends on the speed of displacement of the applicator. When the applicator is moved too quickly, the rate at which the temperature of the skin increases is significantly lower than the rate of temperature increase in the course of “proper” applicator movement speed. A high rate of temperature change is indicative of a static applicator, a condition that may cause burns, blisters and other skin damage. Proper speed of displacement of the applicator could therefore be achieved by controlling the rate of the skin temperature change.

Control of the quality of the RF electrode-to-skin contact for solid and rigid RF electrode/s when such electrodes are applied or coupled to a skin surface covering a “bony” skin area, having minimal fat and muscle tissue, could be achieved by monitoring continuous rate of temperature change, monitoring impedance across the electrodes and monitoring the rate of the impedance change. Implementation of such monitoring potentially includes monitoring impedance alone with further determination of rate of impedance change or in combination with the rate of temperature change.

### BRIEF LIST OF DRAWINGS

The apparatus and the method are particularly pointed out and distinctly claimed in the concluding portion of the specification. The apparatus and the method, however, both as to organization and method of operation, may best be understood by reference to the following detailed description when read with the accompanying drawings, in which like reference characters refer to the same parts throughout the different views. The drawings are not necessarily to scale, emphasis instead being placed upon illustrating the principles of the method.

FIG. 1 is a schematic illustration of an apparatus for personal skin treatment according to an example.

FIGS. 2A and 2B are schematic illustrations of front and side views of an applicator according to an example that in course of operation applies RF energy to a segment of skin.

FIG. 3 is a schematic illustration of the skin (and RF electrodes) temperature dependence on the speed of applicator displacement.

FIGS. 4A and 4B are respectively schematic illustrations of proper and insufficient contact of an RF electrode with a segment of skin.

FIG. 5. is a schematic illustration of the dependence of skin impedance on the quality of electrode-to- skin contact.

FIGS. 6A-6E are schematic illustrations of some examples of the electrodes of the applicator.

FIG. 7A is a front view and FIG. 7B is a side view schematic illustration of another example of the applicator including a skin temperature probe configured to measure the skin temperature and indicate the level of RF energy applied to a segment of skin.

FIGS. 8A and 8B are frontal view illustrations of examples of a rigid electrode to apply or couple RF energy to the skin.

FIG. 9 is an example of a proper rigid RF electrode-to-skin contact quality.

FIG. 10 is a graphic illustration of the skin and/or electrode temperature behavior for a proper rigid RF electrode-to-skin contact quality.

FIG. 11 is an example of a partial rigid RF electrode-to-skin contact.

FIG. 12 is a schematic representation of the rigid RF electrodes being in partial RF electrode-to-skin contact.

FIG. 13 is a graphic illustration of the skin and/or RF electrode temperature behavior for a partial rigid RF electrode-to-skin contact.

FIG. 14 is an example of a rigid RF electrode that in course of displacement over a skin surface covering a “bony” skin is returning to a proper RF electrode-to-skin contact.

FIG. 15 is a graphic illustration of the skin and/or electrode temperature behavior for a rigid RF electrode restoring proper RF electrode-to-skin contact quality.

FIG. 16A is a front view and FIG. 16B is a side view of a schematic illustration of another example of an applicator that in course of operation applies RF energy and optical radiation to a segment of skin.

FIG. 17 is a schematic illustration of an example of an applicator that in course of operation applies ultrasound energy to a segment of skin.

FIG. 18 is a schematic illustration of an example of an applicator that in course of operation applies ultrasound energy and optical radiation to a segment of skin.

FIG. 19 is a schematic illustration of an example of an applicator that in course of operation applies RF energy, ultrasound energy, and optical radiation to a segment of skin.

FIG. 20 is a schematic illustration of an example of an applicator that in course of operation could apply RF energy, ultrasound energy, and optical radiation to a segment of skin formed as a protrusion.

### DETAILED DESCRIPTION OF EXEMPLARY EMBODIMENTS

In the following detailed description, reference is made to the accompanying drawings that form a part hereof. This is shown by way of illustration of different embodiments in which the apparatus and method may be practiced. Because components of embodiments of the present apparatus can be in several different orientations, the directional terminology is used for purposes of illustration and is in no way limiting. It is to be understood that other embodiments may be utilized, and structural or logical changes may be made without departing from the scope of the present method and apparatus. The following detailed description, therefore, is not to be taken in a limiting sense, and the scope of the present apparatus and method is defined by the appended claims.

As used herein, the term “skin treatment” includes treatment of various skin layers such as stratum corneum, dermis, epidermis, skin rejuvenation procedures, wrinkle removal and such procedures as hair removal and collagen shrinking

The term “skin surface” relates to the most external skin layer, which may be stratum corneum, epidermis, or dermis.

As used herein, the term “rate of temperature change” means a change of the skin or electrode temperature measured in temperature units per time unit.

The term “skin heating energy” incorporates RF energy, ultrasound energy, optical radiation, and any other form of energy capable of heating the skin.

As used herein, the term “good quality of the electrode-to-skin contact” relates to firm or almost complete contact between the RF electrode surface and the skin. Contact that does not include voids, air traps, and similar. Good contact quality is defined by almost complete or complete contact between the RF electrode surface and the skin. Good contact facilitates electrical and thermal coupling between the RF electrode surface and the skin. In a similar mode the term “quality of the electrode-to-skin contact” could be related to ultrasound transducers surface-to-skin contact.

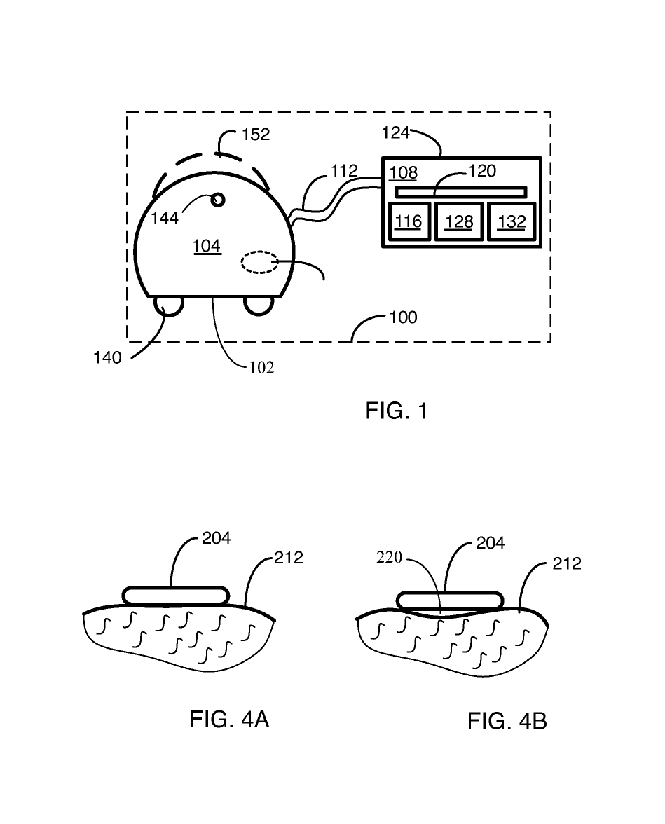

Reference is made to FIG. 1, which is a schematic illustration of an example of the apparatus for safe skin treatment. Apparatus 100 comprises an applicator 104 operative to slide or be displaced along a subject skin (not shown) and apply skin heating energy to the skin from sources of heating energy mounted on surface 102 of the applicator 104 facing the skin, a control unit 108 controlling the operation of apparatus 100, and a harness 112 connecting between applicator 104 and control unit 108. Harness 112 enables electric, fluid, and other type of communication between applicator 104 and control unit 108.

Control unit 108 may include a source of skin heating energy 116, which may be such source as an RF energy generator, a source of optical radiation, or a source of ultrasound energy. Control unit 108 may include control electronics that may be implemented as a printed circuit board 120 populated by proper components. Board 120 may be located, together with control unit 108, in a common packaging 124. Board 120 may include a feedback loop or a mechanism 128 that in course of operation monitors the quality of coupling to the skin of the skin heating energy applied by the applicator and a feedback loop or mechanism 132 for monitoring the temperature of a segment of treated skin and deriving therefrom the rate of temperature change. Apparatus 100 may receive power supply from a regular electric supply network receptacle, or from a rechargeable or conventional battery.

Applicator 104 could include one or a larger number RF energy to skin supplying or coupling electrodes 140, a visual skin treatment progress indicator 144, and an audio skin treatment progress indicator 168. The indicators may be configured to inform or signify to the user the status of interaction of the RF energy with the skin, and alert the user on undesirable applicator displacement speed or RF energy variations. For example, if the applicator displacement speed is slower than the desired or proper displacement speed, an audio process progress indicator will alert or signify the user by way of audio signal. Visual status indicator may be operative to indicate or alert the user with a signal that the applicator displacement speed is higher than the desired displacement speed. Any other combination of audio and visual process progress indicator operation is possible. Feedback loop 128 that in course of operation monitors the quality of coupling to the skin of the skin heating energy may determine the quality of RF electrode-to-skin contact by continuously monitoring the impedance between the electrodes and deriving the impedance rate of change.

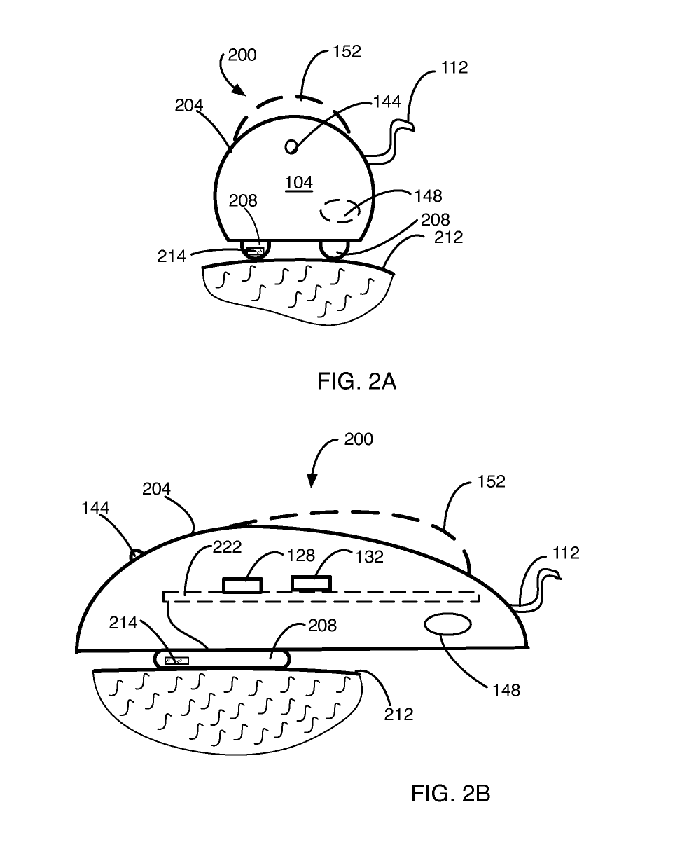

FIGS. 2A and 2B are schematic illustrations of front and side views of an example of an applicator that in course of the operation applies RF energy to a segment of skin. Applicator 200 includes a convenient to hold case 204 incorporating one or a number of electrodes 208 attached to applicator 104 energy applying surface 102 (FIG. 1) and operative to apply safe levels of skin heating energy to a subject skin 212. The skin heating energy in this particular case is RF energy. A temperature sensor such as, for example, a thermistor 214 or a thermocouple is built-in to one or more of electrodes 208 and is configured/operative to provide the electrode temperature reading to a feedback loop 132 operating an RF energy-setting control circuit, which may be implemented as a printed circuit board 222.

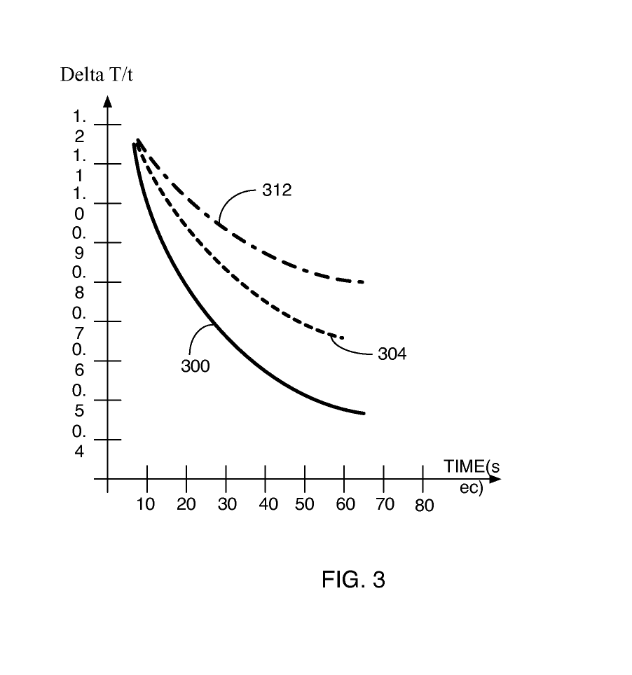

It has been experimentally discovered that the temperature change of the skin segment, located between the RF electrodes and the electrodes in contact with the skin at a constant skin heating energy level, depends on the applicator displacement speed. FIG. 3 schematically illustrates the skin and RF electrodes temperature dependence on the applicator displacement speed. Curve 300 illustrates the rate of temperature change for a static applicator. Curves 304 and 312 illustrate the rate of temperature change as a function of the applicator displacement speed. The applicator displacement speed was respectively 5 cm/sec and 10 cm/sec. (The graphs are given for a thermistor with a negative temperature coefficient.) Other than thermistor temperature detectors such as thermocouples, resistance temperature detectors (RTD), and non-contact optical detectors such as a pyrometer and similar may be employed. The thermistor was selected, since it possesses higher precision within a limited temperature range and a faster response time.

Referring once more to FIGS. 1, 2A and 2B, control circuit 222 includes a mechanism 132 configured to generate a rate of temperature change based on temperature sensor 214 readings. The rate of temperature change may be measured in degrees (Celsius or any other temperature unit) per time unit. Alternatively, there may be a customized integrated circuit including thermistor 214 and a mechanism of converting temperature into the rate of temperature change. Temperature measurement may be converted into a rate of temperature change using either digital or analog conversion circuits.

Heat transfer or coupling from the skin to the RF electrode and accordingly the temperature measured by the temperature sensor is largely dependent on the quality of the contact between the electrode and the skin. Differences in the quality of the contact could cause a large variability in the temperature measurement. Firm or proper quality contact between electrodes 208 and subject skin 212, as illustrated in FIG. 4A, supports proper RF energy and thermal coupling, a short response time of the temperature sensor to the variations in the skin temperature. With poor or improper quality contact, as illustrated in FIG. 4B where, for example, an air pocket 220 is trapped between the electrode 204 and the skin 212, the response time of the temperature sensor may be much longer. In order to improve the RF electrode contact with the skin, a coupling gel is applied to skin 212 improving, to some extent, heat transfer and RF energy coupling. The gel does not completely resolve the problem of or compensate for poor or improper electrode contact bringing about low/poor/improper quality of the electrode—skin contact that could result in increase of skin temperature and lead to skin burns.

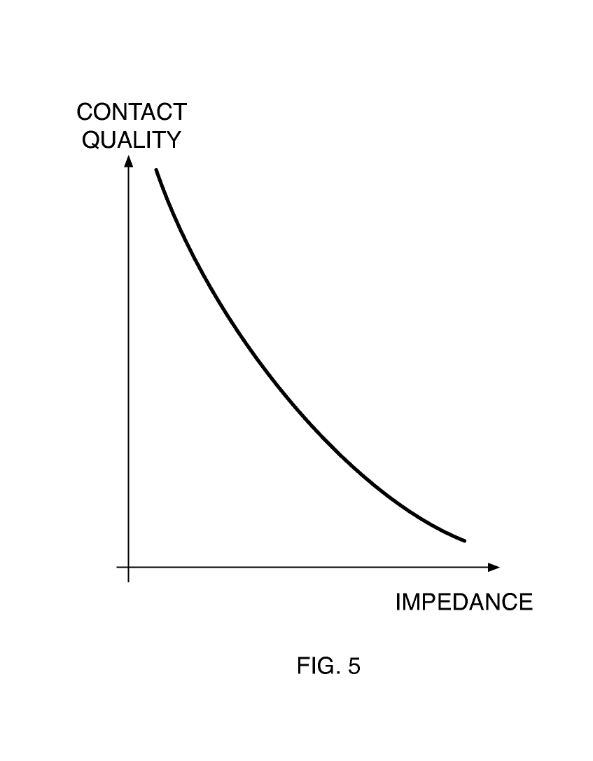

RF energy coupled to the skin induces in the skin an electric current that heats the skin. The current is dependent on the skin impedance, which is a function of the quality of the RF electrode contact with the skin. FIG. 5 is a schematic illustration of the skin impedance dependency on quality of the electrodes with the skin contact. The temperature measured by the sensor is dependent on the actual rate of heat exchange between the electrode and the skin and on the quality of the electrode with the skin contact. Proper contact between electrodes 208 and skin 212 (FIGS. 2A and 2B) may be detected during the treatment by monitoring skin impedance between electrodes 208 as disclosed in the U.S. Pat. No. 6,889,090 to the same assignee. The impedance measurement is an excellent indicator of the electrode-to-skin contact quality. Low impedance between electrodes 208 and skin 212 (FIGS. 2A and 2B) means that a firm or proper contact between the electrode and the skin exists and accordingly the temperature sensor can follow the changes in the skin temperature sufficiently quick. Other known impedance monitoring methods could also be applied.

In addition, it is possible to measure the quality of the thermal contact through the rate of heating (or temperature change) of the temperature sensor, but the measurement would not provide an indication if the rate of heating is indeed rapid or slow, since it may be affected by firm or improper electrode to skin contact. The impedance measurement is independent of the temperature sensor measurements. Continuous impedance monitoring provides electrode to skin contact quality and allows the electrode skin thermal contact influence on the rate of temperature change measurement to be eliminated.

Therefore, control circuit 222 includes a feedback loop or a mechanism 128 (FIG. 2B) operative to continuously monitor the skin impedance by measuring the electric current flowing between electrodes 140 (FIG. 1) or 208 (FIGS. 2A and 2B). Continuous monitoring of the quality of contacts of the electrodes with skin eliminates the influence of the electrode-skin contact on the rate of temperature variations making the rate of temperature variations an objective indicator of the skin RF energy interaction and treatment status.

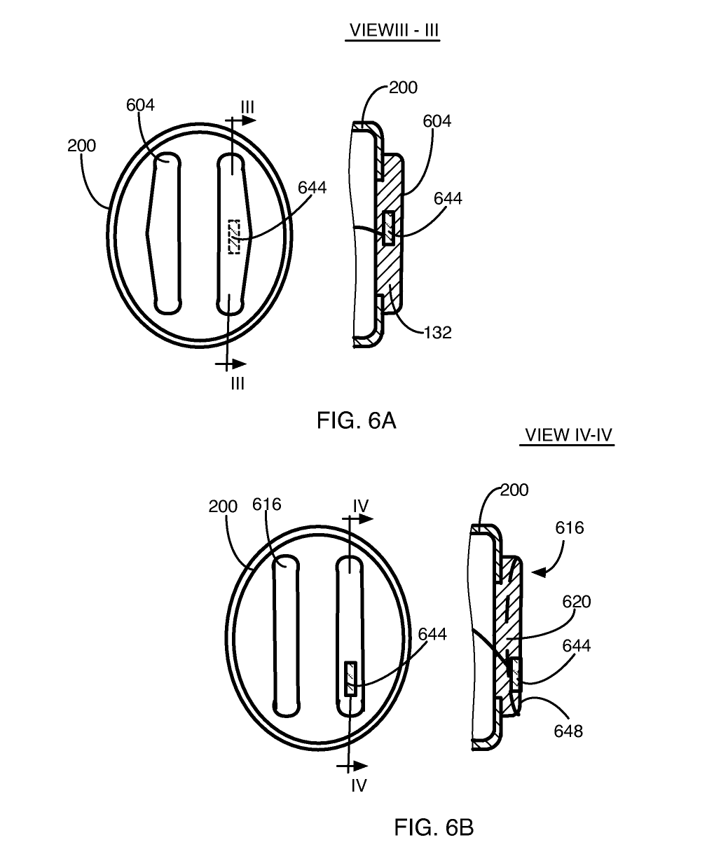

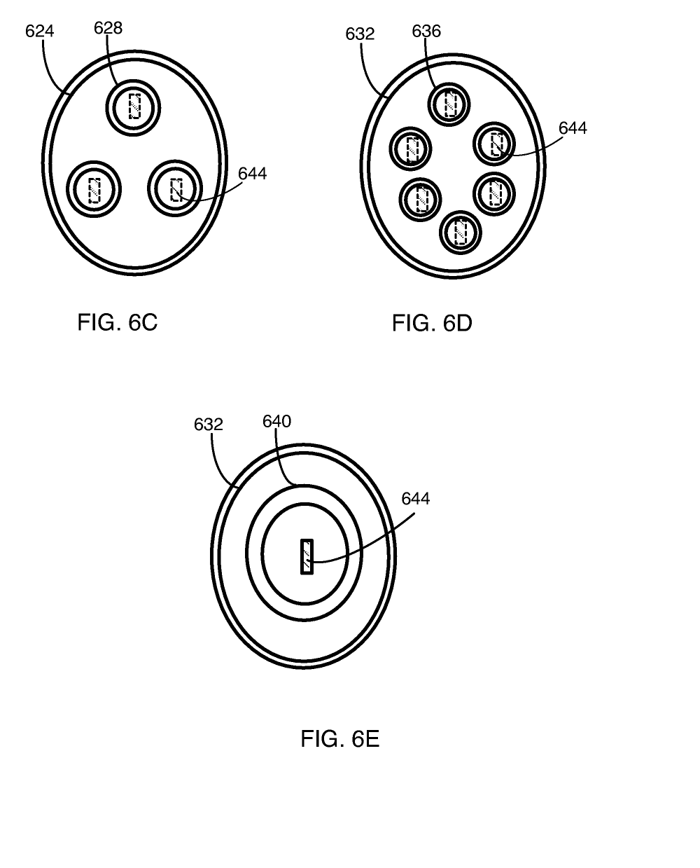

FIGS. 6A, 6B, 6C, 6D and 6E are schematic illustrations of an example of the RF electrodes of the applicator. RF electrodes 604 may be elongated bodies of oval, rectangular or other shape. In one example (FIG. 6A), electrode 604 is a solid electric current conducting body. In another example (FIG. 6B), electrode 616 may be a flexible electric current conducting body. A flexible electrode is capable of adapting its shape, shown by phantom line 620, to the topography of the treated subject skin enabling better contact with the skin. In still a further example, electrode 604 may be a hollow electrode. (A hollow electrode generally has a thermal mass smaller than a comparable size solid electrode.) FIG. 6C shows an applicator 624 containing three equi-shaped electrodes 628. FIG. 6D shows an applicator 632 containing a plurality of equi-shaped electrodes 636. The electrodes may be of round, elliptical, oval, rectangular or other curved shapes, as appropriate for a particular application. The geometry of the electrodes is optimized to heat the skin in the area between the electrodes.

The RF electrodes are typically made of chromium coated copper or aluminum or other metals characterized by good heat conductivity. The electrodes have rounded edges in order to avoid hot spots on the skin surface near the edges of the electrodes. Rounded electrode edges also enable smooth displacement of applicator 104 (FIG. 1) or 204 (FIG. 2) across the skin surface. FIGS. 6A through 6D illustrate bi-polar electrode systems. FIG. 6E illustrates a uni-polar electrode system 640. Each of the electrodes may contain a temperature sensor 644 operative to measure the electrode temperature in course of skin treatment. Temperature sensor 644 may reside inside the electrode or form a continuous plane with one of it surfaces. For example, in FIG. 6B surface 648 forms direct contact with the skin enabling direct skin temperature measurement.

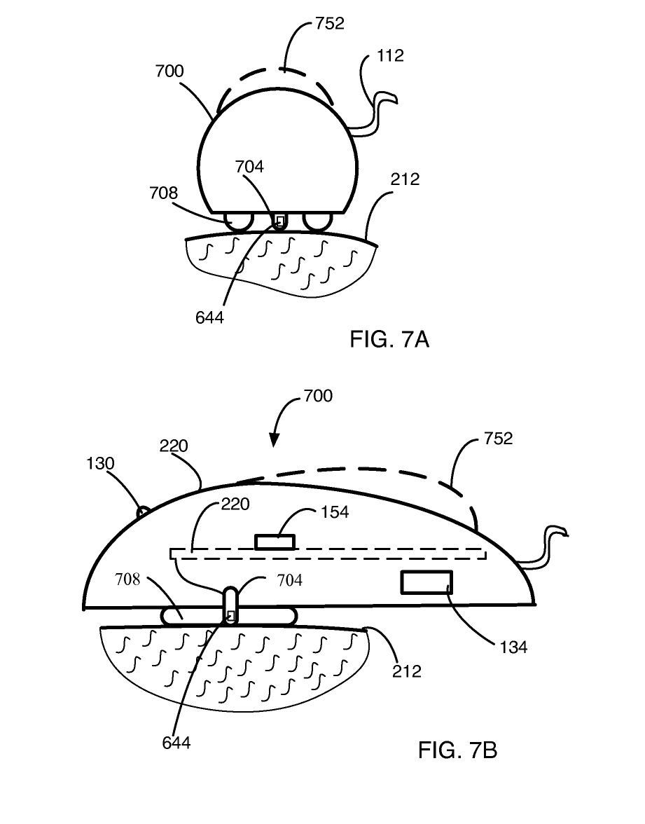

Solid metal electrodes 604 may have a relatively large thermal mass and require time until the correct reading of the temperature sensor 644 is established. FIG. 7A is a front view and FIG. 7B is a side view schematic illustration of another example of an applicator. The temperature sensor 644 may be located in a spring-loaded or fixedly attached probe 704 having a small thermal mass, as compared to the electrodes, and adapted for sliding movement across the subject skin 212. Depending on the size of the skin segment treated, there may be one or more probes 704, with each probe 704 incorporating a temperature sensor 644. Processing of the temperature sensor readings is similar to the processing manner described above and is directed to defining the rate of skin temperature change, or signifying and informing the user of extreme temperature values. Use of an applicator with a number of probes 704 with each probe 704 incorporating a temperature sensor 644 enables a more accurate temperature measurement and rate of temperature change assessment and a uniform treated skin segment thermal profile mapping.

Electrodes 708, of applicator 700 may be coated with a thin metal layer sufficient for RF energy application, wherein the electrodes themselves may be made of plastic or composite material. Both plastic and composite materials are poor heat conductors and a temperature sensor located in such electrodes would not enable rapid enough temperature reading required for RF energy correction and may not provide a correct reading. The addition of a temperature sensor located in a spring-loaded probe or fixedly attached probe 704 allows rapid temperature monitoring even with plastic electrodes. This simplifies the electrode construction and enables disposal where needed of electrodes 708 for treatment of the next subject, and variation of the shape of the electrodes as appropriate for different skin treatments. In an alternative example, the temperature sensor may be an optical non-contact sensor such as a pyrometer.

It is an established practice to apply a coupling gel to the skin before applying the RF energy, to some extent improving heat transfer and RF energy coupling. Accordingly, applicator 700 may include an optional gel dispenser 752 similar or different from gel dispenser 152 (FIGS. 1 and 2). Gel dispenser 752 may be operated manually or automatically. The gel would typically be selected to have an electrical resistance higher than that of the resistance of the skin. In some embodiments a gel reservoir may reside inside control unit 108 (FIG. 1) and be supplied to the skin to be treated with the help of a pump (not shown).

When rigid electrodes are applied and displaced over a skin surface covering a “bony” area having minimal fat and muscle tissue such as for example, forehead, chin, and similar, the contact between the electrode and the skin becomes partial and the quality of the contact deteriorates. When the quality of the contact deteriorates the current density in the remaining contact points grows fast and could cause skin burns.

Because of this it could be good to provide the user with information regarding the change in the quality of RF electrode-to-skin contact and facilitate use of solid and rigid electrodes when applied to a skin surface covering a “bony” area. This could provide a set of features useful for the fast developing field of personal skin treatment apparatuses, features facilitating safe use of personal skin treatment apparatuses, since the typical user of such apparatus may be inexperienced. In case of poor RF electrode-to-skin contact quality the device controller can reduce the output energy to prevent the burns or unpleasant feel.

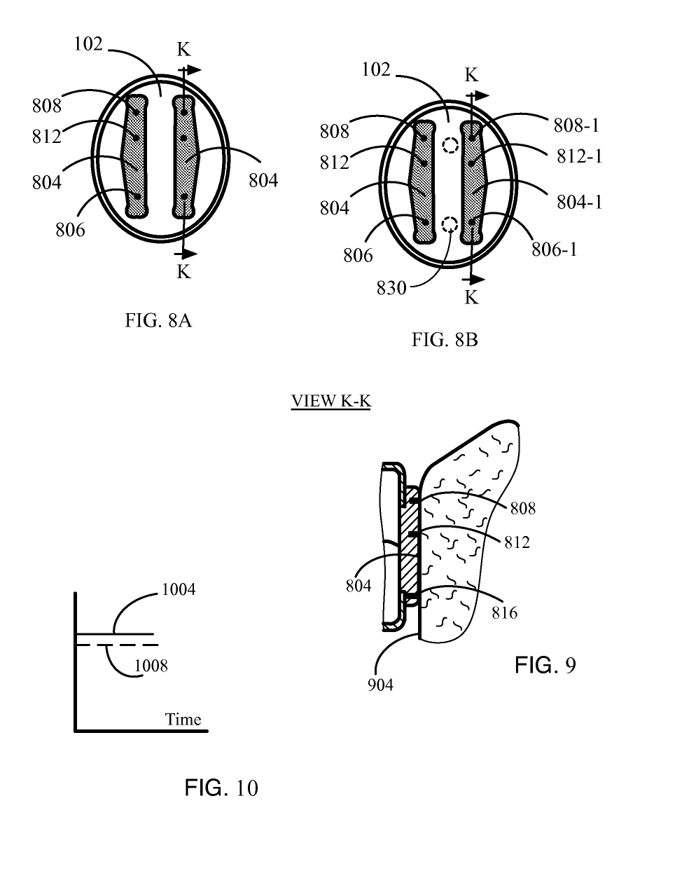

FIG. 8A is frontal view of an example of a rigid electrode to apply or couple RF energy to the skin. RF electrode 804 is mounted on a surface 102 facing the skin of an applicator. Electrode 804 includes three temperature sensors 808, 812, and 806, although more than three or less than three temperature sensors could be incorporated into the RF electrode. Thermistors, thermocouples, and other suitable temperature sensors could be used as such sensors. Alternatively and optionally and as shown in FIG. 8B temperature sensors 808, 812, and 806 may be paired with temperature sensors 808-1, 812-1, and 806-1 located on a second electrode and the temperature differences between each pair of thermistors 808/808-1, 812/812-1 and 806/806-1 measured. Additionally and optionally control circuit 222 feedback loop 132 (FIGS. 1, 2A and 2B) may also be adapted for this purpose. Integration of temperature changes between thermistor pairs 808/808-1, 812/812-1 and 806/806-1, the distance between each pair and measured impedance between the electrodes may contribute to optimization of controller 108 analysis of electrode contact with skin.

In FIG. 8B, thermistor pairs 808/808-1, 812/812-1 and 806/806-1 could be replaced with temperature sensor probes 830. The probes 830 or temperature sensors of the probes, similar to probes 704 as explained above, communicate with control unit 108 and adjust optical radiation intensity as a function of the temperature differences between the temperature sensors.

FIG. 9 is an example of a proper rigid RF electrode-to-skin contact quality. The entire electrode 804 surface is in contact with skin 904. There are no air pockets, voids, or skin folds below the electrode.

FIG. 10 is a graphic illustration of the skin and/or electrode temperature behavior for a proper rigid RF electrode-to-skin contact quality. For comparison purposes FIG. 10 includes also impedance between the RF electrodes behavior. Both impedance 1004 between the RF electrodes being in contact with the skin and skin and/or electrode temperature 1008 are almost constant and do not change, as long as a proper quality of the electrode-to-skin contact is maintained in course of the electrode over the skin displacement.

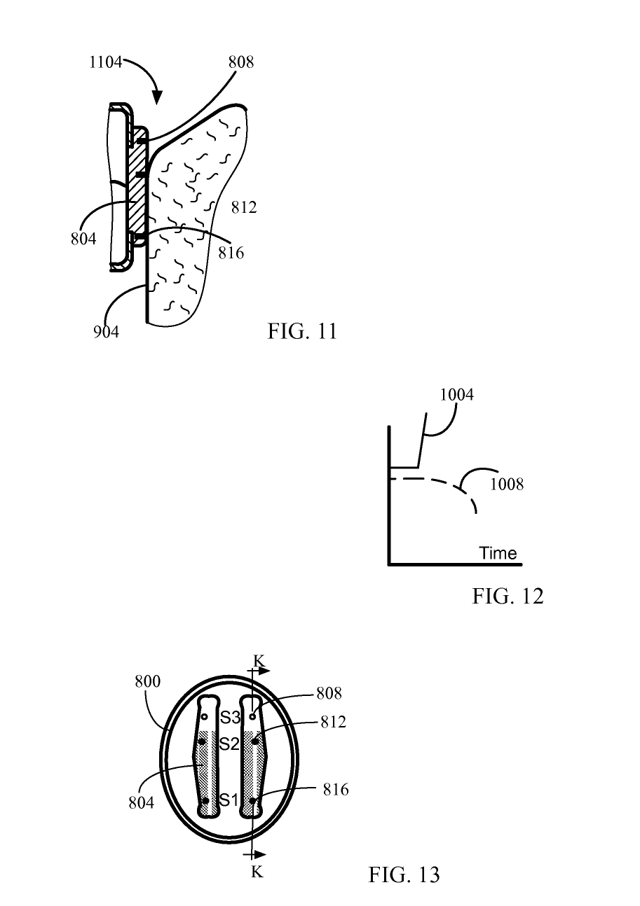

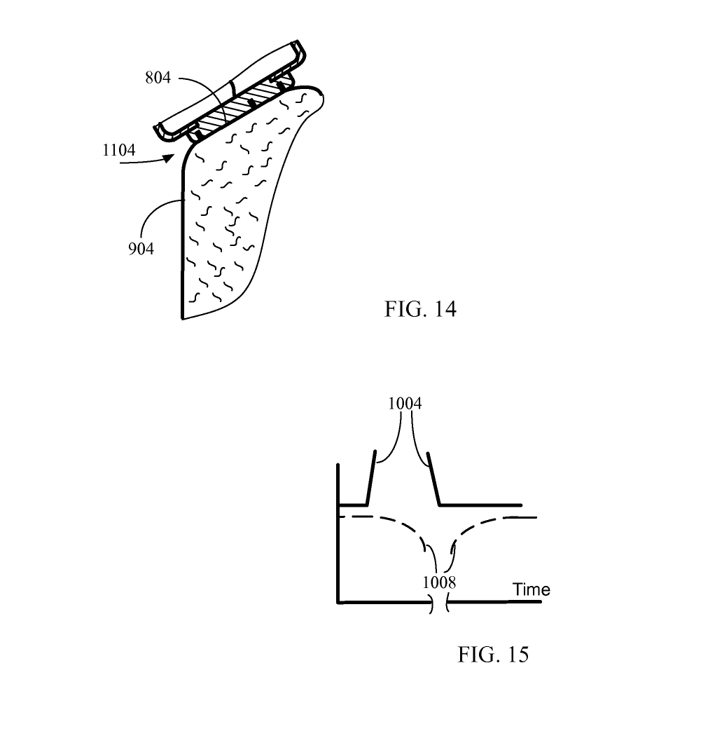

As electrode/s 804 in course of applicator over the skin displacement, slides into a “bony” skin area 1104, as shown in FIG. 11, the contact between the electrode 804 and the skin becomes partial, the temperature of at least of a segment of the electrode (shown in FIG. 13 as clear electrode 804 segment) changes and could become equal to the ambient temperature. Since the RF energy supplied to the electrode remains the same, the value of the RF current density increase and skin 904 temperature and being in contact with it electrode 804 segment (Shown in FIG. 13 as a hatched segment of electrode 804.) grows.

Control unit 108 (FIG. 1) receiving the temperature from the thermistors 808-806 or other temperature sensors could be operative to continuously measure or monitor electrode 804 temperature. In a similar way a number of spring loaded or fixedly attached probes, similar to probe 704 could be operative to continuously measure or monitor the treated skin segment temperature. Based on the received from thermistors 808-816 or other temperature sensors temperature, control unit 108 operates to adjust (reduce or increase) the RF energy supplied to the electrodes and avoid potential skin burns.

Use of two or more temperature sensors mounted on the same electrode, or a number of spring loaded or fixedly attached sensors similar to probe 704, potentially helps to indicate or map which segment of the electrode 804 is out of contact with the skin. In one example, electrode image could be displayed on a display indicating on the segment of the electrode 804 which is out of the contact with the skin. Alternatively, temperature differences between said temperature sensors could be displayed as a map of temperature distribution across the rigid electrode. In another example, a number of LEDs indicating on each of the electrode segments could be used to indicate on a deteriorated contact of a segment of the electrode 804. Indication could be by change of color of the LED or switching it OFF or ON. Based on these indications, the user may undertake corrective steps.

Thermal processes are relatively slow processes and in some instances there could be a longer than desired time delay between the electrodes or skin temperature change and RF energy by control unit 108 adjustments. Impedance between the electrodes changes almost immediately with the changes in RF electrode-to-skin contact quality. Continuous impedance between the electrodes 804 monitoring with proper feedback to control unit 108 could be used for RF energy adjustment as a function of the RF electrode-to-skin contact quality. Controller 108 (FIG. 1) could be operative to continuously monitor impedance and obtain impedance change or rate of impedance change over time and adjust the voltage supply to the electrode in real time. FIGS. 10, 12, and 15 illustrate impedance 1004 between RF electrodes changes as compared to RF electrode or skin temperature changes 1008.

Temperature monitoring and the rate of temperature change could be used alone for RF voltage electrodes supply adjustment. Impedance monitoring and the rate of impedance change could be used alone for RF voltage electrodes supply adjustment. A combination of temperature monitoring and rate of temperature change with impedance monitoring and rate of impedance change could be used for RF voltage to electrodes supply adjustment. Any of the listed above methods of RF voltage supply to electrodes control proper RF electrode-to-skin contact should be taken into account.

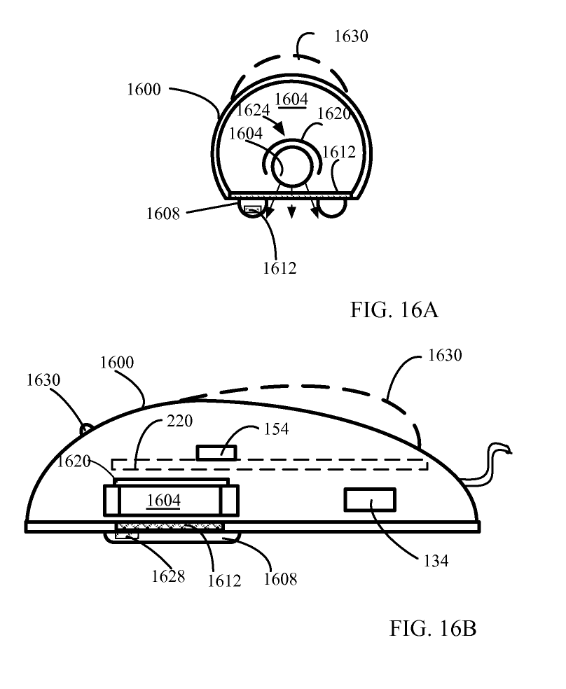

FIG. 16A is a front view and FIG. 16B is a side view schematic illustration of another example of the applicator. Applicator 1600 includes a source of optical radiation 1604 located between electrodes 1608 and operative in course of treatment, to illuminate at least the segment of the skin located between electrodes 1608. The source of optical radiation may be one of a group of sources consisting of incandescent lamps and lamps optimized or doped for emission of red and infrared radiation, and a reflector 1620 directing the radiation to the skin, an LED, and a laser diode. The spectrum of optical radiation emitted by the lamps may be in the range of 400 to 2400 nm and the emitted optical energy may be in the range of 100 mW to 20 W. An optical filter 1612 may be selected to transmit red and infrared or any other portion of light spectrum optical radiation in order to transmit a desired radiation wavelength to the skin. Filter 1612 may be placed between the skin and the lamp and may serve as a mounting basis for one or more electrodes 1608. Reflector 1620 collects and directs radiation emitted by lamp 1604 towards a segment of skin to be treated. When LEDs are used as radiation emitting sources their wavelengths may be selected such as to provide the desired treatment, eliminating the need for a special filter. A single LED with multiple wavelength emitters may also be used.

Operation of the source of optical radiation 1604 at applicable or suitable optical radiation intensity enhances the desired skin effect caused by the RF energy induced current. All RF electrode structures described above, visual and audio signal indicators are mutatis mutandis applicable to respective elements of applicator 1600. A temperature sensor 1628 such as a thermistor, thermocouple or any other suitable temperature sensor, could be incorporated into one or a number of electrodes 1608. A temperature probe or a number of temperature probes (not shown) similar to probe 704 (FIG. 7A and FIG. 7B) may be added and located between the electrodes so as not to mask optical radiation. The probes or temperature sensors of the probes, similar to probes 704 as explained above, communicate with control unit 108 and adjust optical radiation intensity as a function of the temperature differences between the temperature sensors.

A manually or automatically operated gel dispenser 1630 similar to gel dispenser 152 (FIGS. 1 and 2) may be part of the applicator 1600.

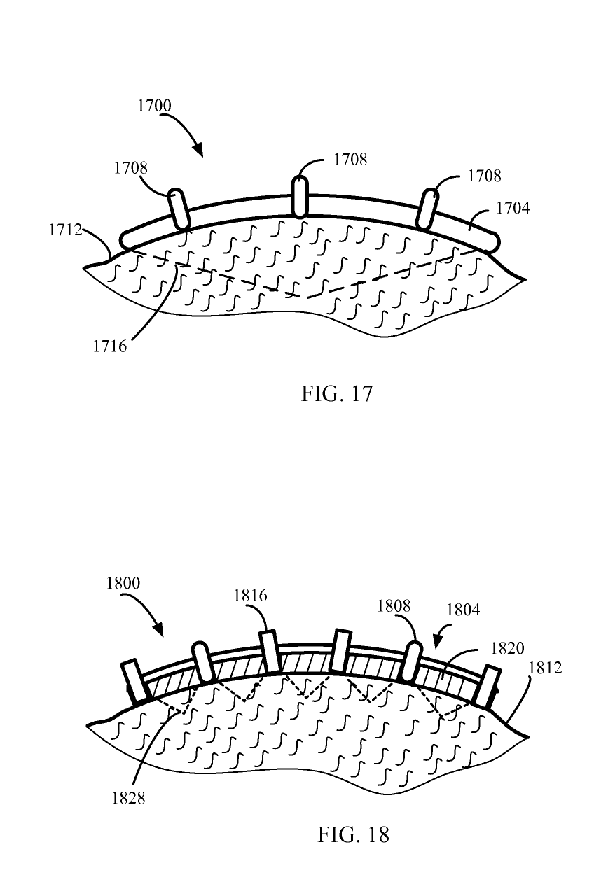

FIG. 17 is a schematic illustration of an example of an applicator that in course of operation applies ultrasound energy to a segment of the skin formed as a protrusion. Ultrasound energy is another type of skin heating energy. The ultrasound energy is applied to the skin of a subject with the help of an applicator 1700, which may include a conventional ultrasound transducer 1704 and one or more temperature probes 1708 arranged to provide the temperature of the treated skin section 1712. Transducer 1704 may be of a curved or flat shape and configured for convenient displacement over the skin. Lines 1716 schematically show skin volume 1712 heated by the ultrasound energy/waves.

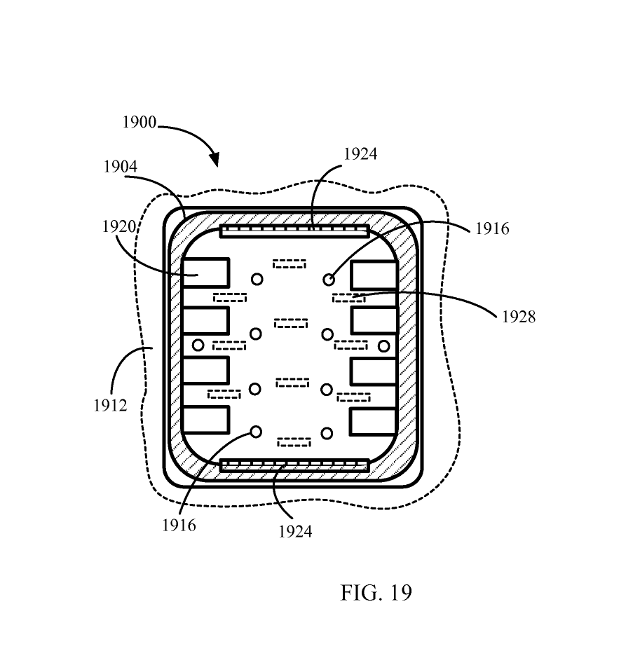

FIG. 18 is a schematic illustration of an example of an applicator that in course of operation applies ultrasound energy and optical radiation to a segment of the skin. The ultrasound energy is applied to skin 1812 of a subject with the help of an applicator 1800, which may include a phased array ultrasound transducer 1804, at least one temperature probe 1808 arranged to provide the temperature of the treated skin segment 1812, and at least one optical radiation source 1816. Individual elements 1820 forming transducer 1804 may be arranged in a desired order and emit ultrasound energy 1824 to heat the desired depth of skin segment 1828. Optical radiation sources 1816 of applicable or suitable optical radiation intensity may be configured to irradiate the same skin segment 1812 treated by ultrasound, accelerating generation of the desired skin effect.

FIG. 19 is a schematic illustration of an example of an applicator that in course of operation applies RF energy, ultrasound energy, and optical radiation to a segment of the skin. FIG. 19 is a top view of the applicator 1900. Applicator 1900 may include one or a larger number of ultrasound wave transducers 1920 operative in course of treatment to apply or couple ultrasound energy to skin 1912, one or few RF voltage supplying electrodes 1924, and one or a larger number of sources 1928 of optical radiation. Applicator 1900 further includes at least one or a number of temperature probes 1916 similar to the earlier described spring loaded of fixed temperature probes. Temperature probes 1916 are in communication with control unit 108 and could operate to adjust ultrasound energy intensity and optical radiation intensity as a function of the temperature differences between the temperature sensors. Ultrasound wave transducers 1920 are configured to cover as large as possible segment of skin 1912. RF energy supplying electrodes 1924 could be arranged to provide a skin heating current in the direction perpendicular to that of propagation of ultrasound energy. Presence of firm or proper contact of skin 1912 with electrodes 1924 may be detected, for example, by measuring the skin impedance. Firm or proper contact of skin 1912 with ultrasound wave transducers 1920 could be detected by measuring the power of reflected from skin 1912 ultrasound energy.

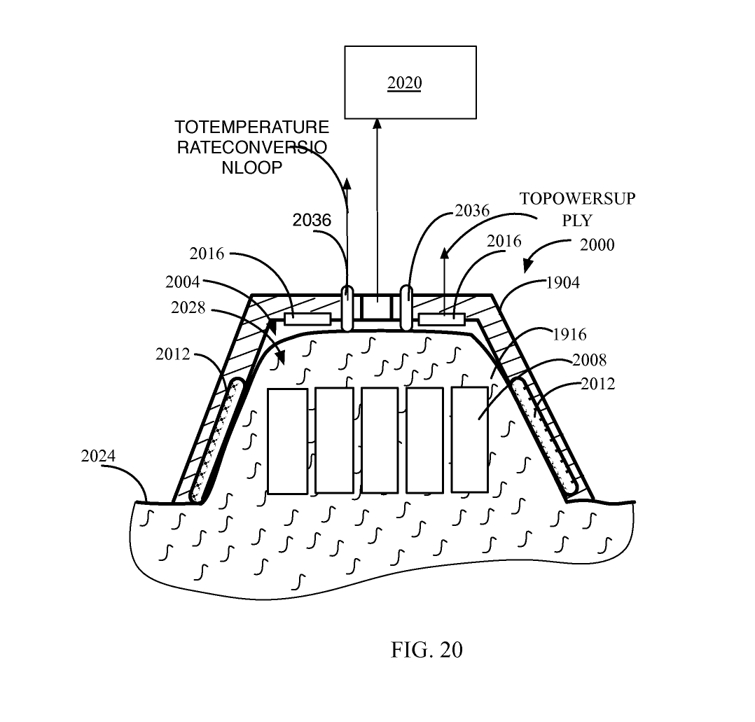

FIG. 20 is a schematic illustration of an example of the present applicator operative to apply in course of treatment RF energy, ultrasound energy, and optical radiation to a segment of the skin formed as a protrusion. Applicator 2000 is a bell shaped case with inner segment 2004 containing one or more ultrasound wave transducers 2008, one or more RF energy supplying electrodes 2012 and optionally one or more sources 2016 of optical radiation. A vacuum pump 2020 is connected to the inner volume 2004 of applicator 2000. When applicator 2000 is applied to skin 2024, the inner segment 2004 becomes hermetically closed. Operation of vacuum pump 2020 evacuates air from inner segment 2004. Negative pressure in inner segment 2004 draws skin 2024 into inner segment 2004 forming a skin protrusion 2028. As skin protrusion 2028 grows, it occupies a larger volume of inner segment 2004, and spreads in a uniform way inside the segment. The protrusion spreading enables firm contact of skin 2024 with electrodes 2012. When firm contact between skin protrusion 2028 and electrodes 2012 is established, RF energy is supplied to skin protrusion 2028. Presence of firm contact of skin 2024 with electrodes 2012 may be detected for example, by measuring the skin protrusion 2024 impedance, as explained hereinabove.

Applicator 2000 further includes one or few ultrasound wave transducers 2008 operative to couple ultrasound energy to skin protrusion 2024. Ultrasound transducers 2008 could be conventional transducers or phased array transducers.

Applicator 2000 and other applicators described may contain additional devices supporting skin and electrodes cooling, auxiliary control circuits, wiring, and tubing not shown for the simplicity of explanation. A thermo-electric cooler or a cooling fluid may provide cooling. The cooling fluid pump, which may be placed in a common control unit housing.

For skin treatment procedures the user couples the applicator to a segment of skin, activates one or more sources of skin heating energy and applies or couples the energy supplied by the sources of skin heating energy to the skin. For example, applying RF energy or ultrasound energy to skin, or irradiating the skin with optical radiation. RF energy interacts with the skin inducing in the skin a current that heats at least the segment of the skin located between the electrodes. The heat produces the desired effect on the skin, which may be wrinkle removal, hair removal, collagen shrinking or destruction, and other cosmetic and skin treatments. In order to improve RF to skin coupling the treated skin segment may be first coated by a layer of suitable gel typically having resistance higher than that of the skin.

Ultrasound energy causes skin cells mechanical vibrations. Friction between the vibrating cells heats the skin volume located between the transducers and enables the desired treatment effect, which may be body shaping, skin tightening and rejuvenation, collagen treatment, removal of wrinkles and other aesthetic skin treatment effects.

Application of optical radiation of proper wavelength to skin causes an increase in skin temperature, since the skin absorbs at least some of the radiation. Each of the mentioned skin heating energies may be applied to the skin alone or in any combinations of them to cause the desired skin effect.

For skin treatment the user or operator continuously displaces the applicator across the skin. When the user displaces the applicator at a speed slower than the desired or proper speed, an audio signal indicator alerts user attention and avoids potential skin burns. The temperature sensor continuously measures temperature and may shut down RF energy supply when the rate of temperature increase or change is too fast or when the absolute temperature measured exceeds the preset limit. When the user displaces the applicator at a speed higher than the desired or proper speed, the rate of temperature change is slower than desired. The visual signal indicator alerts user attention and avoids formation of poorly treated or under-treated skin segments. This maintains the proper efficacy of skin treatment.

The applicator may be configured to automatically change the RF energy coupled to the skin. In such mode of operation, where the applicator is displaced at an almost constant speed, a controller based on the rate of temperature change and/or on impedance and/or impedance rate of change may automatically adjust the value or magnitude of RF voltage coupled to the skin. For example, at a high rate of temperature change the magnitude of RF energy coupled to the skin will be adapted and reduced to match the applicator displacement speed. At lower rates of temperature change, the magnitude of RF energy coupled to the skin will be increased to match the applicator displacement speed. The user or operator may be concurrently alerted in a manner disclosed hereinabove. In a similar manner, temperature monitoring could be used to alert the user or automatically adjust the ultrasound power or light intensity or a combination of all of them to ensure a desired treatment result. This mode of operation also maintains the proper efficacy of skin treatment.

A number of embodiments have been described. Nevertheless, it will be understood that various modifications may be made without departing from the spirit and scope of the method. Accordingly, other embodiments are within the scope of the following claims:

1. An apparatus for personal skin treatment with skin heating energy, said apparatus comprising:
at least one rigid electrode, mounted on a surface of an applicator facing the skin, said electrode being at least partially in contact with a “bony” segment of a skin of a subject and operative to apply RF voltage to the skin and measure skin impedance and wherein the rigid electrode includes at least two temperature sensors;
an RF energy generator operative to supply said rigid electrode with RF energy; and
a control unit communicating with said RF generator and including a mechanism operative to continuously monitor skin impedance between the electrodes and calculate rate of change of monitored skin impedance and adjust RF energy supplied to said rigid electrode as a function of skin impedance and rate of change of the skin impedance; and
when the at least one rigid electrode is at least partially in contact with a “bony” segment of a skin of a subject the two temperature sensors measure different temperature.

2. The apparatus according to claim 1, wherein said control unit includes a mechanism that based on said monitored skin impedance or the rate of change of the skin impedance determines quality of the RF electrode-to-skin contact.

3. The apparatus according to claim 2, wherein said control unit adjust supply of the RF energy to the rigid electrode as a function of the quality of the RF electrode-to-skin contact.

4. The apparatus according to claim 1, wherein said applicator also includes at least two temperature sensors located on said rigid electrode and when the rigid electrode is at least partially in contact with a “bony” segment at least one of the two temperature sensors measures ambient temperature.

5. The apparatus according to claim 1, wherein said applicator also includes at least two temperature sensors each located on a probe.

6. The apparatus according to claim 1 wherein said controller includes a mechanism operative to monitor the difference in the temperature between said temperature sensors compare said difference with a predetermined protocol and accordingly adjust RF energy supply to said rigid electrode.

7. The apparatus according to claim 6, wherein said mechanism operative to monitor the difference in the temperature is operative to calculate rate of temperature change and based on said rate of temperature change adjust RF energy supply to said rigid electrode.

8. The apparatus according to claim 6, wherein said controller based on the difference in the temperature provided by the mechanism operative to monitor the difference in the temperature between said temperature sensors displays which segment of the electrode is out of contact with the skin.

9. The apparatus according to claim 1 further comprising:
at least one source of optical radiation operative to irradiate and heat the skin between the rigid electrodes;
at least one spring loaded or fixedly attached temperature sensor operative to measure skin temperature and provide the measurements to a mechanism operative to monitor temperature differences between said temperature sensors; and
wherein said control unit adjust optical radiation intensity as a function of said temperature differences between the temperature sensors.

10. The apparatus according to any one of claims 1 and 9 further comprising:
at least one source of ultrasound energy operative to couple said energy and heat the skin between the rigid electrodes;
at least one spring loaded temperature sensor operative to measure skin temperature and provide the measurements to a mechanism operative to monitor temperature differences between said temperature sensors; and
wherein said control unit adjust ultrasound energy intensity as a function of said temperature differences between the temperature sensors.

11. The apparatus according to claim 1 further comprising:
at least one visual signal indicator operative to signify a user of quality of electrode-to-skin contact and display a map of rigid electrode temperature distribution; and
at least one audio signal indicator operative to signify a user on quality of the electrode-to-skin contact.

12. A method of user-controlled efficacy of skin heating energy application to skin, said method comprising:
coupling to the skin an applicator having at least one rigid RF electrode, a visual signal indicator, at least one audio signal indicator, and a source of RF energy and wherein the rigid electrode includes at least two temperature sensors and an LED display;
applying said energy to said skin;
displacing the applicator across the skin and monitoring at least skin impedance changes and calculating rate of skin impedance changes; and
based on said skin impedance changes and the rate of skin impedance changes indicating on partial electrode-to-skin contact and wherein the LED display displays which segment of the electrode is out of contact with the skin.

13. The method according to claim 12 further comprising adjusting the RF energy supplied to said rigid RF electrode as a function of the partial electrode-to-skin contact.

14. The method according to claim 12, wherein also
monitoring temperature differences between at least two temperature sensors located on said rigid electrode;
comparing said differences with a predetermined protocol; and
accordingly adjusting RF energy supply to said rigid electrode.

15. The method according to claim 12 wherein the temperature sensors are paired with temperature sensors located on a second electrode to measure the temperature differences between each pair of temperature sensors.

16. The apparatus according to any one of claim 1, further comprising:
at least one source of ultrasound energy operative to couple said energy and heat the skin between the rigid electrodes;
at least one spring loaded temperature sensor operative to measure skin temperature and provide the measurements to a mechanism operative to monitor temperature differences between said temperature sensors; and
wherein said control unit adjust ultrasound energy intensity as a function of said temperature differences between the temperature sensors.
